SEQUENCE LISTING (8 sequences)

SEQ ID NO: 1 - protein, 7 aa, Artificial Sequence
  Artificial Sequence - Synthetic
YSPWTNF

SEQ ID NO: 2 - protein, 7 aa, Artificial Sequence
  Artificial Sequence - Synthetic
YSPWTNF

SEQ ID NO: 3 - protein, 7 aa, Artificial Sequence
  Artificial Sequence - Synthetic
YSPWTNF

SEQ ID NO: 4 - protein, 7 aa, Artificial Sequence
  Artificial Sequence - Synthetic
YSPWTNF

SEQ ID NO: 5 - protein, 4 aa, Artificial Sequence
  Artificial Sequence - Synthetic
YSPW

SEQ ID NO: 6 - protein, 4 aa, Artificial Sequence
  Artificial Sequence - Synthetic
YSPW

SEQ ID NO: 7 - protein, 7 aa, Staphylococcus aureus
YSPCTNF

SEQ ID NO: 8 - protein, 5 aa, Artificial Sequence
  Artificial Sequence - Synthetic
PCTNF

(19) United States
(12) Patent Application Publication
(10) Pub. No.: US 2003/0050248 A1
(43) Pub. Date: Mar. 13, 2003

(54) Novel amino acid and peptide inhibitors of Staphylococcus virulence

(76) Inventors: Susan C. Wright, Saratoga, CA (US); James W. Larrick, Woodside, CA (US); Yuqiang Wang, Mountain View, CA (US)

Correspondence Address: MERCHANT & GOULD PC, P.O. BOX 2903, MINNEAPOLIS, MN, 55402-0903, US

(21) Appl. No.: 09/780,967

(22) Filed: Feb. 9, 2001

Related U.S. Application Data
(60) Provisional application No. 60/181,629, filed on Feb. 10, 2000.

(51) Int. Cl. A61K 38/08; C07K 7/06

(52) U.S. Cl. 514/17; 530/329

(57) ABSTRACT

Isolated synthetic modified amino acids and peptides, which inhibit of the production of virulence factors in bacteria, particularly Staphylococcus, are described. The amino acids and peptides can be used in a method for treatment or prevention of Staphylococcal infections or can be integrated into a medical device to inhibit the development of infection associated with the use of such devices.

This application claims priority to U.S. provisional application No. 60/181,629, filed Feb. 10, 2000, which is hereby incorporated by reference in its entirety.

### FIELD OF THE INVENTION

This invention relates to inhibitors of the production or expression of virulence factors in Staphylococcus and methods of treating or preventing bacterial infection using the same.

### REFERENCES

Arthur M. & Courvalin P., Genetics and mechanisms of glycolipid resistance in enterococci. Antimicrob, Agents & Chemother, 37:1563-1571, 1993.

Atherton E. and Sheppard R. C., “Solid Phase Peptide Synthesis” IRL Press at Oxford University Press 1989.

Balaban N. & Novick R. P., Autocrine regulation of toxin synthesis by S. aureus, Proc. Natl Acad. Sci. USA 92:1619-1623, 1995.

Balaban N, Goldkorn T., Nhan R. T., Dang L. B., Rasooly A., Wright S. C., Larrick J. W., Carlson J. R., Autoinducer of virulence as a target for vaccine and therapy against Staphylococcus aureus, Science 280: 438-440, 1998.

Barrett J. F., Goldschrnidt R. M., Lawrence L. E., Foleno B., Chen R., Demers J. P., Johnson S., Kanojia R., Fernandez J., Bernstein J., Licata L., Donetz A., Huang S., Hlasts D. J., Macielag M. J., Ohemeng K., Frechette R., Frosco M. B., Klaubert D. H., Whiteley J. M., Wang L., & Hoch J. A., Antibacterial agents that inhibit two-component signal transduction systems. Proc. Natl Acad. Sci. 95:5317-5322, 1998.

Bernheimer A. W., Assay of hemolytic toxins, Methods Enzymol. 165:213-217, 1988.

Bodansky et al., Chem. Ind. (London) 38, 15997, 1966.

Bunce C., Wheeler L., Reed G., Musser J. and Barg N., Murine model of cutaneous infection with gram-positive cocci, Infect. Immun. 60:2636-2640, 1992.

Dann E. J., Weinberger M., Gillis S., Parsonnet J., Shapiro M. & Moses A. E., Bacterial laryngo-tracheitis associated with toxic shock syndrome in an adult, Clin. Inf. Dis. 18:437-439. 1994.

Gould I. M., Risk factors for acquisition of multiply drug-resistant gram-negative bacteria, Euro. J. Clinic. Microbiol. Infect. Dis. 13 Suppl 1:S30-8, 1994.

Hiramatsu K., Aritaka N., Hanaki H., Kawasaki S., Hosoda Y., Hod S., Fukuchi Y., & Kobayashi L., Dissemination in Japanese hospitals of strains of Staphylococcus aureus heterogenously resistant to vancomycin, Lancet 350:1670-1673, 1997.

Ji G., Beavis R. C. and Novick R. P., Cell density control of Staphylococcal virulence mediated by an octapeptide pheromone, Proc. Natl Acad. Sci. (USA) 92:12055-12-59, 1995.

Ji G., Beavis R. & Novick R. P., Bacterial interference caused by autoinducing peptide variants, Science 276:2027-2030, 1997.

Jones J., “The Chemical Synthesis of Peptides”, Clarendon Press, Oxford 1994.

Lambe D. W., Ferguson K. P., & Keplinger J. L., Pathogenicity of Staphylococcus lugdunensis, Staphylococcus schieffed, and three other coagulase-negative staphylococci in a mouse model and possible virulence factors, Can. J. Microbiol. 36:455-463, 1990.

Lina G., Jarraud S., Ji G., Greenland T., Pedraza A., Etienne J., Novick R. P., and Vandenesch F., Transmembrane topology and histidine protein kinase activity of AgrC, the agr signal receptor in Staphylococcus aureus, Molec. Micro. 28:655-662, 1998.

Lowry F. D. Staphylococcus aureus infections, New Engi. J. Med. 339:520-532, 1998.

Mayville P., Ji G., Beavis R., Yang H., Goger M., Novick R. P., & Muir T. W., Structure-activity analysis of synthetic autoinducing thiolactone peptides from Staphylococcus aureus responsible for virulence, Proc. Natl Acad. Sci. 96:1218-1223, 1999.

Mamo W., Jonsson P., Flock J. I., Lindberg M., Muller H. P, Wadstrom T. & Nelson L., Vaccination against S. aureus mastitis: immunological response of mice vaccinated with fibronectin-binding protein (FnBP-A) to challenge with S. aureu, Vaccine 12:988-991, 1994.

Mekalanos J. J., Environmental signals controlling expression of virulence determinants in bacteria, J. Bacteriol. 174:1-7, 1992.

Merrifield R. B., J. Am. Chem. Soc. 85, 2149-2154, 1963.

Morley J. S., Trends Pharm Sci. pp. 463-468, 1980.

Moses A. E., Mevorach D., Rahav G., Sacks T., Simhon A. & Shapiro M,). Group A Streptococcus bacteremia at the Hadassa Medical Center in Jerusalem. Clin. Inf. Dis. 20:1393-1395, 1995.

Nishiuchi Y et al., Tet. Lett. 37(42):7529-7532, 1996.

Noble W. C. Virani Z. & Cree R. G., Co-transfer of vancomycin and other resistance genes from Enterococcus faecalis NCTC 12202 to Staphylococcus aureus, FEMS Microbiol. Lett. 93:195-198, 1992.

Novick R. P. Ross H., Projan S. J., Komblum J., Kreiswirth B. & Moghazeh S., Synthesis of Staphylococcal virulence factors is controlled by a regulatory RNA molecule, EMBO J. 12:3967-3875, 1993.

Novick R. P., Projan S. J., Komblum J., Ross H. F., Kreiswirth B., Vandenesch F. and Moghazeh S., The agr P2 operon: an autocatalytic sensory transduction system in Staphylococcus aureus, Mol. Gen. Genet. 248:446-458, 1995.

Otto M., Supmuth R., Vuong C., Jung G., and Gotz F., Inhibition of virulence factor expression in Staphylococcus aureus by the Staphylococcus epidermidis agr pheromone and derivatives, FEBS Left. 450:257-262, 1999.

Panlilio A. L., Culver D. H., Gaynes R. P., Banerjee S., Henderson T. S., Tolson J. S. & Marton W. J., Methicillin-resistant S. aureus in US hospitals, 1975-1991, Inf. Contr. & Hosp. Epidem. 13:582-586, 1992.

Patti J. M., Bremell T., Krajewska-Pietrasik D., Abdelnour A., Trakoski A., Ryden C. & Kook M., The S. aureus collagen adhesin is a virulence determinant in experimental septic arthritis, Infect. Immun. 62:152-161, 1994.

Pearson J. P., et al., Proc. Natl Acad. Sci. USA 92:1490-1494, 1995.

Pietta and Marshall, Chem. Comm., 650, 1970.

Pittet D. & Wenzel R. P., Arch. Intern. Med. 155:1177-1184, 1995.

Rappuoli R., et al., AN OVERVIEW OF BACTERIAL SIGNAL TRANSDUCTION. IN: SIGNAL TRANSDUCTION AND BACTERIAL PATHOGENESIS. Scarlato V., Arico B. and Rappuoli R. (eds). Springer Verlag, Heidelberg, pp. 1-4, 1995.

Roychoudhury S. et al., Proc. NatI. Acad. Sci. USA 90:965-969, 1993.

Silver L. L. & Bostian K. A., Antimicrobial Agents Chemotherapy. 37:377-383, 1993.

Silverstein S. C. & Steinberg T. H., in MICROBIOLOGY, eds. B. D. Davis, R. Dulbecco, H. N. Eisen & H. S. Ginsberg, Lippincott, Philadelphia, pp. 485-506, 1990.

Spatola A. F., Vega Data, Vol.1, Issue 3, March 1983,

Spatola A. F., CHEMISTRY AND BIOCHEMISTRY OF AMINO ACIDS, PEPTIDES AND PROTEINS, B. Weinstein, ed., Marcel Dekker, New York, p. 267, 1983

Tegmark K., et al., J. Bacteriol. 180:3181-3186, 1998.

Thakker et al., Infect Immun 66(11):5183-9, 1998.

Vandenesch F., et al., FEMS Microbiol. Lett. 111: 115-122, 1993.

### BACKGROUND OF THE INVENTION

Staphylococci are gram positive bacterial pathogens which cause a wide variety of diseases ranging from superficial abscesses (boils, styes and furuncles) and other localized abscesses; deeper infections such as osteomyelitis, pneumonia, endocarditis, urinary tract infections, septic arthritis, meningitis, post-operative wound infections, septicemia and food poisoning. S. aureus is a major cause of hospital acquired (nosocomial) infection of surgical wounds and S epidermidis, causes infections associated with indwelling medical devices. (See, e.g., Silverstein et al., 1990; Patti et al., 1994; Dann et al., 1994.)

Multiple antibiotic resistance is increasingly common in S. aureus and S. epidermidis. Hospital strains of Staphylococcus are often resistant to many different antibiotics and strains resistant to most clinically useful drugs, apart from the glycopeptide antibiotics, vancomycin and teicoplanin, have been described. In the hospital setting, especially tertiary-care facilities, the incidence of drug-resistant gram-positive infections is increasing (Silver et al., 1993; Pittet et al., 1995; Moses et al., 1995). Increased use of antibiotic prophylaxis in immunosuppressed and intensive care patients is likely to aggravate the problem, as is the use of new broad-spectrum agents in the community (Gould, 1994).

S epidermidis nosocomial isolates are also often resistant to several antibiotics including methicillin. In addition, S aureus expresses resistance to antiseptics and disinfectants, such as quaternary ammonium compounds, which may aid its survival in the hospital environment.

For serious hospital infections with multi-drug resistant S. aureus, vancomycin is the only currently effective antibiotic. Vancomycin resistance is carried by conjugative plasmids that can be transferred to S. aureus in a laboratory setting (Noble et al., 1992) and has already appeared naturally in enterococci (Arthur et al., 1993). Furthermore, the appearance of intermediate vancomycin resistance in S. aureus has already been reported in the USA (Lowry, 998) and Japan (Hiramatsu et al., 1997). Vaccines have been developed targeting the organism or exotoxins it produces, but these approaches have not always been successful (Mamo et al., 1994), underscoring the urgent need to develop new methods to control Staphylococcal infections.

Similar to other gram positive bacteria, S. aureus causes disease chiefly through the production of virulence factors such as hemolysins, enterotoxins and toxic shock syndrome toxin, which facilitate the survival, multiplication and spread of the organism in infected tissue (Mekalanos, 1992). The synthesis of most virulence factors in S. aureus is controlled by the accessory global regulon (agr) locus, which is activated by secreted autoinducing peptides (AIPs) (Novick et al., 1993; Novick et al., 1995).

The presence of agr and regulation of virulence by RNAIII has been demonstrated in all strains of S. aureus tested to date as well as several other species of staphylococci including S. epidermidis, S. lugdunesis, S. hemolyticus (Vandenesch et al., 1993), and S. warneri (Tegmark et al., 1998). Therefore, a universal inhibitor of RNAIII synthesis may have broad therapeutic applications for infections caused by diverse strains of staphylococci.

In addition to those mechanisms described for staphylococci, similar regulatory mechanisms involving autoinducers have been described for other bacterial species (Rappuoli et al., 1995).

Despite impressive successes in controlling or eliminating bacterial infections by antibiotics, the widespread use of antibiotics both in human medicine and as a feed supplement in poultry and livestock production, has led to drug resistance in many pathogenic bacteria.

Accordingly, Staphylococcal infection remains a medical concern, it would therefore be desirable to provide a treatment method which shows efficacy in reducing the incidence and severity of Staphylococcal infection, and is well tolerated by patients, with minimal or no side effects. It would also be desirable to provide improved therapeutic compounds and compositions for carrying out the method.

### SUMMARY OF THE INVENTION

It is therefore a general object of the invention to provide compositions and methods for treatment of bacterial infection which is associated with virulence factor production.

The invention provides an isolated synthetic peptide inhibitory of Staphylococcal virulence selected from the group consisting of Fmoc-TyrSerPro(modifiedTrp)ThrAsnPhe, wherein the modifiedTrp is an S-phenylthiocarbamate derivative of tryptophan (SEQ ID NO:2); TyrSerPro(modifiedTrp)ThrAsnPhe, wherein the modifiedTrp is an S-phenylthiocarbamate derivative of tryptophan (SEQ ID NO:3); Fmoc-TyrSerPro (modifiedTrp) ThrAsnPhe, wherein the modifiedTrp has a BOC group linked to the ring nitrogen (SEQ ID NO:4); Fmoc-TyrSerPro(modifiedTrp), wherein the modifiedTrp is a phenylthiocarbamate derivative of typtophan (SEQ ID NO:5); Fmoc-TyrSerPro(modifiedTrp), wherein the modifiedTrp has a BOC group linked to the ring nitrogen (SEQ ID NO:6) and pharmaceutical compositions comprising such peptides.

The invention further provides a method of preventing or treating Staphylococcus infection in a subject, by administering to the subject a therapeutically effective amount of an isolated synthetic peptide as described above or by administering to the subject a therapeutically effective amount of an isolated synthetic amino acid inhibitory of Staphylococcal virulence of the structure, (Fmoc)NH—CHR—COOH, wherein Fmoc is a 9-fluorenylmethoxycarbonyl group and R is a non-polar side chain.

Exemplary isolated synthetic amino acids inhibitory of Staphylococcal virulence include Fmoc-L-Trp(Boc)-OH, Fmoc-D-Trp(Boc)-OH, FMoc-2-aminobenzoic acid, Fmoc-1-amine-cyclohexane carboxylic acid, (R,S)-Fmoc-3-amino-1-cyclohexane carboxylic acid, Fmoc-D-tetrahydroisoquinoline-1-carboxylic acid, Fmoc-4-bromo-L-phenylalanine, Fmoc-4-chloro-L-phenylalanine, Fmoc-5-phenyl-pyrrolidine-2-carboxylic acid, (R)-Fmoc-4-amino-5-phenyl-pentanoic acid, Fmoc-L-His(Trt)-OH, Fmoc-L-1,2,3,4-tetrahydronorharman-3-carboxylic acid, FMoc-2-L-styrylalanine, FMoc-2-L-Lys(Z)-OH, Fmoc-4-methyl-L-phenylalanine and Fmoc-L-Leu-OH.

The Staphylococcus infection under treatment may involve an antibiotic resistant strain of Staphylococcus, e.g., one that is methicillin-resistant or vancomycin-resistant.

The method of treatment may include administration of an anti-bacterial amino acid or peptide of the invention together with an antibiotic or integrated with a medical device.

These and other objects and features of the invention will be more fully appreciated when the following detailed description of the invention is read in conjunction with the accompanying drawings.

### BRIEF DESCRIPTION OF THE FIGURES

FIG. 1A depicts the native sequence peptide, Tyr-Ser-Pro-Trp-Thr-Asn-Phe or YSPWTNF (“PEP”);

FIG. 1B depicts the sequence of a synthetic VIF peptide, Fmoc-Tyr-Ser-Pro-(modifiedTrp)-Thr-Asn-Phe, wherein modifiedTrp is a phenylthiocarbamate derivative of tryptophan (“VIF-1”, SEQ ID NO:2);

FIG. 2 depicts a synthetic Trp analog with an Fmoc group at the N terminus and a t-BOC group linked to the ring nitrogen (“Fmoc-Trp(Boc)-OH” or “FTB”).

### DETAILED DESCRIPTION OF THE INVENTION

I. Definitions

Generally, the nomenclature used herein, and the laboratory procedures in bacterial and cell culture and protein chemistry are those that are well known and commonly employed in the art. Unless otherwise defined, all technical and scientific terms used herein have the same meaning as commonly understood by one of ordinary skill in the art to which this invention belongs.

The amino acid sequence of the peptides of the present invention is indicated in the usual manner for peptides or proteins, using the conventional one-letter or three-letter codes for the naturally occurring amino acids and abbreviations for other amino acids as provided herein, and written with the amino terminus at the left and the carboxy terminus at the right, with adjacent amino acids being joined typically through normal amide, or “peptide,” bonds.

“Fmoc” is the abbreviation for a 9-fluorenylmetboxycarbonyl group. “Boc” is the abbreviation for a tert-butoxycarbonyl group. When the abbreviation precedes the three letter or one letter amino acid code, it indicates that the substituent is at the α-amino group, e.g., Fmoc-Trp is N-α-Fmoc-tryptophan; when the abbreviation is placed in parentheses following the three letter or one letter code, it indicates that the substituent is on the side chain, e.g., Boc-Lys(Fmoc) is N-α-Boc-N-ε-Fmoc-lysine.

As used in a method of treatment herein, a “therapeutically effective amount” of an amino acid or peptide composition of the present invention is an amount that is sufficient to achieve a clinically significant reduction of a Staphylococcus infection, preferably a reduction of at least 30%, more preferably a reduction of at least 50%, most preferably a reduction of at least 90% as measured by any conventional technique, for example, the Bunce model which measures the reduction in size of a cutaneous lesion.

As used in a method of prevention herein, a “therapeutically effective amount” of the amino acid or peptide composition of the present invention is an amount that is sufficient to prevent or significantly decrease the occurrence of clinically significant symptoms of Staphylococcus infection, in a situation in which infection would normally be expected to occur.

As used herein, “pharmaceutically acceptable” refers to molecular entities and compositions that are physiologically tolerable and do not typically produce an allergic or similar untoward reaction, for example, gastric upset, dizziness and the like, when administered to a human. A pharmaceutically acceptable carrier encompasses any of the standard pharmaceutically accepted carriers well known to those of ordinary skill in the pharmaceutical arts. Suitable carriers can be found for example in Remington's Pharmaceutical Sciences (18^{th} Edition, Mack Publishing Co., Easton Pa., (1990)). Typical carriers include, for example, pastes, powders, ointments, jellies, waxes, oils, lipids, semi-solid gels, phosphate buffered saline solution, water emulsions such as an oil/water emulsion or a triglyceride emulsion. A more detailed description of pharmaceutical acceptable carriers and other formulations is provided elsewhere herein.

“VIF peptide” is a virulence inhibitory factor peptide. Similarly, a “VIF amino acid” is a virulence inhibitory factor amino acid, e.g., modified tryptophan or “modTrp”. VIF peptides and amino acids of the present invention inhibit the production of Staphylococcus virulence factors and/or are active in an RNAIII induction assay, and/or show inhibitory activity in an vivo model for Staphylococcus infection.

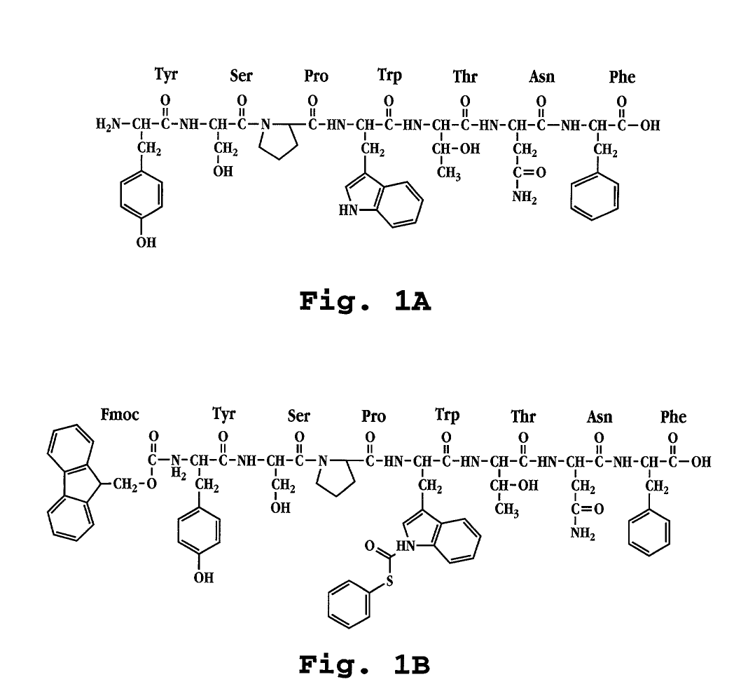

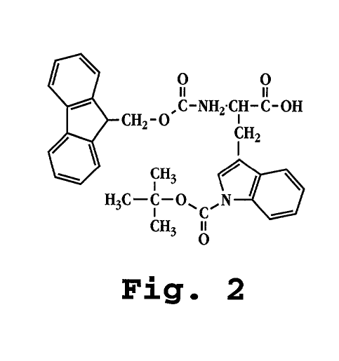

“VIF-1” is the particular VIF peptide having the structure depicted in FIG. 1B, that is, (Fmoc-Tyr)-Ser-Pro-Trp(phenylthiocarbamate)-Thr-Asn-Phe.

By “inhibit the production of Staphylococcus virulence factors” is meant a compound is active to decrease the production of one or more Staphylococcus virulence factors by at least 20% in one or more in vitro or in vivo assays.

By “Staphylococcal virulence” is meant a decrease in the production of one or more Staphylococcus virulence factors by at least 20% or a reduction in Staphylococcal pathogenicity in vivo.

As used herein, an “anti-bacterial amino acid or peptide”, refers to an amino acid or peptide which shows an inhibitory activity of at least 20% (e.g., a 20% reduction) in one or more in vitro assays for Staphylococcus virulence factor production and/or RNAIII induction or show inhibitory activity in an vivo model for Staphylococcus infection.

By “analog” is intended a peptide having activity as a virulence inhibitory factor and an amino acid sequence that differs from the sequence YSP(modW)TNF by one or two conservative amino acid substitutions.

“RIP” is the naturally-occurring RNAIII inducing peptide produced by bacterial strain RN833, which has been recently shown to be a heptapeptide which forms a cyclic thiolactone structure having the amino acid sequence TyrSerProCysThrAsnPhe.

“PEP” is the synthetic heptapeptide described by Balaban et al. (1998) having the unmodified amino acid sequence TyrSerProTrpThrAsnPhe.

By “isolated” when referring to the amino acids or peptides of the invention is intended a particular amino acid or peptide molecule substantially separated from other macromolecules or components that would have been present or created during the synthesis and/or modification of the peptide. For amino acid and peptides that are synthesized or modified entirely by chemical techniques, an isolated amino acid or peptide is one that has been substantially separated from other amino acids or peptides that may have been generated during the synthesis or modification reactions.

For peptides that are synthesized at least in part by recombinant techniques, the term also includes a peptide that has been substantially removed from cellular components, e.g. DNA, RNA, proteins, that may have been present during the recombinant synthesis. An “isolated” peptide is one that has been purified to substantial homogeneity. Typically, a peptide is “isolated” if it comprises at least 80% of the total number of peptides and other macromolecules in the sample. For some applications, the isolated peptide will comprise at least 90%, preferably at least 95%, of the total number of peptides and other macromolecules in the sample.

By “synthetic” when referring to the amino acid and peptides of the present invention is intended an amino acid or peptide that has, at least in part, been synthesized or modified by chemical techniques in vitro. Synthetic peptides include peptides that are synthesized and modified entirely by chemical techniques as well as peptides that are synthesized recombinantly, for example in a cellular system, purified and modified by chemical techniques in vitro.

“ModTrp”, “modified Trp” and “modW” all refer to the modified tryptophan residues that are useful in the peptides of the present invention as detailed herein.

II. Autoinducing Peptides (“RIP”) and Virulence Inhibitory Factor (“VIF”) Peptides

agr autoinducing peptides (AIPs) of staphylococci have been reported that are peptides of 7 to 9 amino acid residues, and vary in amino acid sequence among different stains, with the exception of a conserved cysteine residue five amino acids from the C-terminus. AIP propeptides have been reported to undergo posttranslational modification to form a cyclic structure containing a thiolactone bond linking the conserved cysteine and the C-terminal amino acid (Mayville et al., 1999). Based on activity, S. aureus strains have been placed in at least four different groups, Groups I-IV (Ji et al., 1997). Each of the AIPs can activate the agr response within the same group and inhibit the agr response in strains belonging to different groups, acting by suppressing expression of virulence factors, rather than by inhibiting cell growth.

Several studies have described the modification of AIPs from Staphylococcus with the goal of producing therapeutic inhibitors. The thiolactone bond and cyclic structure were indicated as essential for the agr activating function of synthetic S. aureus AIPs (Mayville et al., 1999).

In one study, a modified AIP based on the sequence of the S. epidermidis AIP was shown to inhibit production of virulence factors by S. aureus group I (Otto et al., 1999), and in another study, an inhibitor termed “RNAIII inhibitory peptide” or “RIP” (Balaban and Novick, 1995), purified from RN833 culture supernatants by reverse phase HPLC (Balaban et al., 1998), was shown to have the sequence TyrSerProXaaThrAsnPhe, where Xaa was an unknown amino acid. Two synthetic peptides ProCysThrAsnPhe (peptide 1) and TyrSerProTrpThrAsnPhe (peptide 2 or “PEP”) were synthesized based on the RIP sequence and peptide 2 was reported to inhibit induction of RNAIII as effectively as RIP purified from Staphylococcal supernatant (Balaban, et al., 1998), while synthetic peptide 1 was inactive in the same assay.

In accordance with the present invention, it has been discovered that peptides having the sequence, TyrSerPro(modified Trp)ThrAsnPhe or YSP(modW)TNF have activity in Staphylococcus virulence factor production and RNAIII induction.

It has been further discovered that modified amino acids also exhibit inhibitory activity in an RNA III activity assay and in assays for α-toxin (α-hemolysin) and toxic shock syndrome toxin (TSST) production. (See Example 2.) Thus the present invention also includes derivatives of single amino acids.

Unlike the naturally-occurring AIPs that have been reported to date, which are cyclic peptides, the VIF peptides of the present invention are linear peptide molecules.

In addition, comparative studies with the peptides and amino acid described herein indicate that the unmodified synthetic PEP having the sequence YSPWTNF (FIG. 1A) has no significant activity as an inhibitor of Staphylococcus virulence factor production or RNAIII induction.

A. Structure of the VIF Amino Acids and Peptides.

A VIF amino acid of the present invention inhibits the production of Staphylococcus virulence factors and/or is active in an RNAIII induction assay, and/or shows inhibitory activity in an vivo model for Staphylococcus infection.

The isolated synthetic anti-bacterial amino acids of the invention generally have the structure: (Fmoc)NH—CHR—COOH, where Fmoc is a 9-fluorenylmethoxycarbonyl group and R is a non-polar side chain.

More specifically, such isolated synthetic anti-bacterial amino acids have a free carboxyl group (COOH), an Fmoc group substituted alpha amino group and a non-polar group side chain (exemplified by the relative activity of Fmoc-L-Ala-OH, IC₅₀=6.5 μg; Fmoc-L-Phe-OH, IC₅₀=1.3 μg; Fmoc-L-Val-OH, IC₅₀=4.2 μg; Fmoc-L-azetidine-2-carboxylic acid, IC₅₀=12 μg; racemic Fmoc-trans-3-phenyl-azetidine-2-carboxylic acid, IC₅₀=3.9 μg; Fmoc-beta-cyclohexyl-L-alanine, IC₅₀=4.0 μg and Fmoc-3-(2-naphthyl)alanine, IC₅₀=4.2 μg).

The results presented herein demonstrate that (1) a polar group decreases activity (exemplified by the relative activity of Fmoc-L-Phe-OH, IC₅₀=1.3 μg; Fmoc-L-Trp-OH, IC₅₀=9 μg; Fmoc-D-Trp(Boc)-OH, IC₅₀=1.0 μg; Fmoc-L-3-Pyridylalanine, not active; and Fmoc-beta-(2-thienyl)-alanine, IC₅₀=25 μg); (2) a natural amino acid is not required for activity (exemplified by the relative activity of Fmoc-L-Ala-OH, IC₅₀=6.5 μg; racemic Fmoc-trans-3-phenyl-azetidine-2-carboxylic acid IC₅₀=3.9 μg; (2S,5R)-Fmoc-5-phenyl-pyrrolidine-2-carboxylic acid, IC₅₀=1.0 μg; (R)-Fmoc-4-amino-5-phenyl-pentanoic acid, IC₅₀=1.7 μg; (R,S)-Fmoc-3-amino-3-(4-bromophenyl)-propionic acid, IC₅₀=1.0 μg; and Fmoc-1-amino-1-cyclohexane carboxylic acid, IC₅₀=0.9 μg); and (3) a D or L form has no effect on activity (exemplified by the relative activity of Fmoc-L-Trp(Boc)-OH, IC₅₀=1.2 μg; Fmoc-D-Trp(Boc)-OH, IC₅₀=1.0 μg; Fmoc-L-phenylglycine, IC₅₀=-5.0 μg and Fmoc-D-phenylglycine, IC₅₀=5.5 μg).

Exemplary synthetic anti-bacterial amino acids include Fmoc-L-Trp(Boc)-OH, Fmoc-D-Trp(Boc)-OH, FMoc-2-aminobenzoic acid, Fmoc-1-amine-cyclohexane carboxylic acid, (R,S)-Fmoc-3-amino-1-cyclohexane carboxylic acid, Fmoc-D-tetrahydroisoquinoline-1-carboxylic acid, Fmoc-4-bromo-L-phenylalanine, Fmoc-4-chloro-L-phenylalanine, Fmoc-5-phenyl-pyrrolidine -2-carboxylic acid, (R)-Fmoc-4-amino-5-phenyl-pentanoic acid, Fmoc-L-His(Trt)-OH, Fmoc-L-1,2,3,4-tetrahydronorharman-3-carboxylic acid, FMoc-2-L-styrylalanine, FMoc-2-L-Lys(Z)-OH, Fmoc-4-methyl-L-phenylalanine and Fmoc-L-Leu-OH. (See Table 4.)

Suitable N-terminal α-amino derivatives are exemplified by Fmoc α-amino derivatives, while other N-terminal α-amino derivatives may also be effective.

An anti-bacterial amino acid of the invention may be a L or D amino acid and may be of natural or synthetic form.

In one exemplary embodiment, the anti-bacterial amino acid is a modified tryptophan (“modTrp”) having a modification at the indole nitrogen of the Trp, for example, S-phenylthiocarbamate or Boc. The N-terminal amino acid of the VIF peptide is typically modified at the α-amino group. Other minor modifications of tryptophan, for example at the indole nitrogen are possible provided that the modifications does not compromise the inhibitory activity of the amino acid.

A VIF peptide of the present invention is a linear peptide having one of the following amino acid sequences: Fmoc-TyrSerPro(modifiedTrp)ThrAsnPhe, wherein the modifiedTrp is an S-phenylthiocarbamate derivative of tryptophan (SEQ ID NO:2); Fmoc-TyrSerPro (modifiedTrp) ThrAsnPhe, wherein the modifiedTrp has a BOC group linked to the ring nitrogen (SEQ ID NO:4); TyrSerPro(modifiedTrp)ThrAsnPhe, wherein the modifiedTrp is an S-phenylthiocarbamate derivative of tryptophan (SEQ ID NO:3); Fmoc-TyrSerPro(modifiedTrp), wherein the modifiedTrp is a phenylthiocarbamate derivative of tryptophan (SEQ ID NO:5); Fmoc-TyrSerPro(modifiedTrp), wherein the modifiedTrp has a BOC group linked to the ring nitrogen (SEQ ID NO:6); FMOC-modifiedTrp (“Fmoc-Trp(Boc)-OH”) wherein the modifiedTrp has a Boc group linked to the ring nitrogen; Trp(Boc)-OH, wherein the modifiedTrp has a Boc group linked to the ring nitrogen; Fmoc-Trp; Fmoc-modTrp wherein the modifiedTrp is a phenylthiocarbamate derivative of tryptophan and mod-Trp, wherein the modifiedTrp is a phenylthiocarbamate derivative of tryptophan.

The modified tryptophan (“modTrp”) includes modifications at the indole nitrogen of the Trp, for example S-phenylthiocarbamate or Boc. The N-terminal amino acid of the VIF peptide can also be modified at the α-amino group or can be unmodified. Suitable N-terminal α-amino derivatives include N-Fmoc derivatives. Other minor modifications of the amino acid residues, for example at side chain functional groups of the VIF peptides are also possible provided that the modifications do not compromise the inhibitory activity of the peptides.

In one preferred embodiment, a VIF peptide of the present invention comprises the peptide Fmoc-TyrSerPro-Trp (S-phenylthiocarbamate)-ThrAsnPhe (SEQ ID NO:2). The peptides of the invention typically comprise L-amino acids selected from those amino acids naturally occurring in proteins but may comprise D-amino acids and/or amino acids other than the genetically encoded amino acids. The peptides of the invention comprise amino acids, as described above, in a linear polymer, joined by amide, or “peptide”, bonds typical of naturally occurring peptides. However, one or more peptide bonds may be replaced by substitute linkages such as those obtained by reduction or elimination. Thus, one or more —CONH— peptide linkages can be replaced with other types of linkages such as —CH₂NH—, CH₂S—, —CH₂CH₂—, —CH═CH— (cis or trans), —COCH₂—, —CH(OH)CH₂—, or CH₂SO—, by methods known in the art (see, for example, Spatola A. F., 1983; Spatola, A. F., 1983; Morley J. S., 1980).

The present invention also includes pharmaceutically acceptable salts of the VIF amino acids and peptides described herein. Such salts would include organic or inorganic addition salts, including hydrochloride, hydrobromide, phosphate, sulfate, acetate, succinate, ascorbate, tartrate, gluconate, benzoate, malate, fumarate, stearate and pamoate salts.

III. Inhibitory Activity of the VIF Amino Acids and Peptides

The amino acid and peptides of the present invention are useful as inhibitors of the induction or synthesis of RNAIII in Staphylococcus bacteria, in particular, RNAIII induction or synthesis by any of a number of Staphylococcus strains, including S. aureus, S. epidermidis, S. warneri and S. hemolyticus. The VIF amino acids and peptides of the invention are also useful as inhibitors of production of virulence factors by Staphylococcus species. Without limitation to any one particular theory, it is likely that inhibition of the production of virulence factors is effected through an inhibition of the production of RNAIII. Inhibition of the production of virulence factors results in suppression or diminution of infection by Staphylococcus bacteria. The peptides of the present invention are therefore useful in the treatment and prevention of Staphyloccoccal infections, particularly S. aureus, S. epidermidis, S. warneri or S. hemolyticus infections.

The effectiveness of a VIF amino acid or peptide of the present invention in the treatment or prevention of Staphylococcal infection can be determined in a number of ways. For example, by examining the ability of the peptide to inhibit the production of RNAIII or virulence factors, e.g. α-toxin (α-hemolysin), by the bacteria, or by studying inhibition of Staphylococcal infection in an animal model. The amino acids and peptides of the present invention were assayed for RNAIII inhibitory activity. This may be accomplished by any of a number of assays that are well known in the art or described in detail herein. The assay may measure inhibition of RNAIII production directly, for example, by Northern analysis of bacterial RNA or by way of an RNAIII-reporter fusion construct, or indirectly, for example, by assaying for inhibition of virulence factor production or inhibition of Staphylococcal infection in vivo. Evaluation of the activity of the anti-bacterial amino acids and peptides of the invention are described In Example 2 (in vitro assays) and Example 3 (in vivo assays).

An RNAIII: βlactamase fusion construct (rnaiii:blaZ), for measuring the induction of RNAIII synthesis calorimetrically, has been described (Ji et al., 1995). The fusion construct is introduced into a Staphylococcal host, for example S. azireus RN6390B, by methods that are well known in the art. As the bacteria carrying the fusion construct grow to the late exponential phase, they release AIPs which then autoinduce the synthesis of RNAIII resulting in the synthesis of β-lactamase. The βlactamase activity in the supernatant can be measured calorimetrically. Addition of a VIF amino acid or peptide to the growth medium will inhibit the induction of RNAIII synthesis by the endogenous AIP, resulting in a decrease in β-lactamase activity compared to a control without the amino acid or peptide. A VIF amino acid or peptide of the invention will produce at least a 20% decrease, preferably a 50% or greater decrease in β-lactamase activity compared to a control without the amino acid or peptide. Typically, the assay is carried out as follows. In a microtiter plate, mid-exponential S. aureus cells containing the rnaiii:blaZ fusion construct are incubated in CY broth with shaking at 37° C. for about 50 min together with the VIF amino acid or peptide to a final concentration of amino acid or peptide of between about 0.01 to 50 μg/ml. Sodium azide is added to a concentration of about 0.02% together with a β-lactamase substrate (for example, nitrocefin; 50 μl at 132 μg/ml in 0.1 M sodium phosphate, pH 5.8). The samples are read in a microtiter plate reader at 500 rim at different time points from 1-30 min. after addition of the substrate An increase in OD₅₀₀ of 0.001/min. equals 1 unit of β-lactamase activity, as described previously (Ji et al, 1995).

Alternatively, RNAIII levels in the presence and absence of VIF amino acid or peptides can be assayed in a Northern blot analysis using ³²β-labeled RNAIII probes as described in Ji et al., 1995 and Novick et al., 1993.

A further assay useful for evaluating the activity of a VIF amino acid or peptide involves a determination the downstream products of RNAIII induction, that is, the virulence factors, for example, Staphylococcal α-toxin (α-hemolysin) or toxic shock syndrome toxin (TSST). The inhibitory activity of a VIF amino acid or peptide is demonstrated by a decrease in the amount of virulence factor produced by the bacteria in the presence of the VIF amino acid or peptide compared to a control which lacks the amino acid or peptide. The virulence factors can be assayed in any of a number of ways that are well known in the art or described herein. For example, the production of virulence factors can be determined by an assay that measures the presence or activity of the virulence factor. In general, in carrying out such assays the Staphylococcal strain that produces the particular virulence factor to be assayed is grown in CY broth with shaking at 37° C. starting in early exponential phase at 3×10⁸/ml. A VIF amino acid or peptide is added to the culture. RNAIII is transcribed after 2-3 hr. of culture under these conditions followed by production of virulence factors. At different time points ranging from about 2-7 hr., samples of supernatant are removed to test for virulence factors, e.g., a hemolytic toxins, such as Staphylococcal α-toxin, the detection of which has been described using rabbit erythrocytes (Bernheimer, 1988). Typically, the assay is carried out as follows: Erythrocytes suspended in an appropriate buffer are incubated with different dilutions of a Staphylococcal supernatant at 37° C. for 30 min. The cells are then pelleted and the concentration of hemoglobin in the supernatant is estimated by reading the absorbance at 545 nm. The amount of hemoglobin released is proportional to the amount of α-toxin in the bacterial supernatant. The addition of detergent is used to determine 100% release of hemoglobin. Although this assay is highly sensitive to α-toxin, it may also detect other hemolysins released by the bacteria.

The presence or absence of virulence factors can alternatively be determined in an immunoassay, for example, using an ELISA. An ELISA has been developed using purified antibodies directed against toxic shock syndrome toxin 1 (TSST-1), as well as purified TSST-1 to generate a calibration curve (with both reagents obtained from Toxin Technology, Inc., Sarasota, Fla.). Similar ELISA assays can be developed for other virulence factors. Antibodies specific for other virulence factors are known, or can be developed by methods that are well known in the art. An exemplary ELISA is carried out as follows. Microtiter plates are coated with a solution of antibodies (polyclonal or monoclonal) directed against TSST-1 (or any of the other virulence factors) at about 4.0 μg/ml in PBS. The plates are incubated overnight at room temperature and washed several times with water. Wells are then filled with blocking buffer (for example, 0.05% Tween 20) and incubated 30 min. at room temperature, followed by additional water rinses. A test sample (e.g., 0.05 ml of a bacterial supernatant suspected of containing the virulence factor) is then added and incubated 2 hr. at room temperature. A standard curve is prepared by adding known amounts of purified virulence factor as a reference point for quantitation of the Staphylococcal supernatants. Purified antibodies directed against the virulence factor can be conjugated to biotin using a commercially available kit (Pierce) and added to the plate at 1.0 μg/ml diluted in blocking buffer. After incubating 2 hours at room temperature, the plate is rinsed again with water, streptavidin conjugated to alkaline phosphatase is added and incubated 2 more hours. The plate is rinsed and a chromogenic alkaline phosphatase substrate (e.g., p-nitrophenyl phosphate) is added, then after sufficient time to allow color development, the OD is read at 405 nm in a plate reader.

The VIF amino acids and peptides of the present invention may also be assayed for activity in an in vivo animal model of Staphylococcus infection. For example, the murine model described by Thakker et al., 1998. Typically, a mouse treated with a VIF amino acid or peptide of the invention using such an in vivo animal model will have at least a 20%, preferably a 50% or greater increase in survival time relative to a control mouse which is not treated with a VIF amino acid or peptide. (See Example 3.)

IV. Synthesis/Production of VIF Amino Acids and Peptides

The peptides of the present invention can be conveniently synthesized by any conventional peptide synthesis method, such as by solid phase peptide synthesis, as described by Merrifield, 1963 and Atherton et al., 1989, by solution phase synthesis as described by Jones, 1994, or by both solid- and solution-phase methods, as known in the art. A useful review of solid phase synthetic methods can be found in Solid-Phase Peptide Synthesis, Methods in Enzymology, Fields, G. Ed., Vol. 289, 1997.

A typical solid-phase peptide synthesis starts from the C-terminal end of peptide. A suitable starting material can be prepared, for example, by attaching the required protected alpha-amino acid to a chloromethylated resin, a hydroxymethylated resin, a benzhydrylamine resin (BHA), or to a para-methylbenzhydrylamine resin (p-Me-BHA). As an example, an available chloromethylated resin is BIOBEADS.RTM. SX 1 by BioRad Laboratories, Richmond, Calif. The preparation of the hydroxymethyl resin is described by Bodansky et al., 1966. The BHA resin is described by Pietta et al., 1970 and is commercially available by Peninsula Laboratories Inc., Belmont, Calif.

Briefly, the C-terminus of the growing peptide is covalently bound to a solid support, or resin, during synthesis. Three chemical reactions are repeated for each amino acid that is added to the peptide chain: deprotection, activation, and coupling. During the process amino acids are “protected”, or derivatized to prevent unwanted reactions at their alpha-amino and side-chain functionalities. Following addition to the peptide, the protecting group is removed (“deprotection”) to make the alpha-amino group on the end of the peptide chain accessible, followed by “activation”, which converts the next amino acid to be added to an active ester and “coupling” when the active ester forms an amide bond with the deprotected alpha-amino group on the end of the peptide chain. After coupling, a new cycle of synthesis begins with the next deprotection. When automated synthesis is complete, side-chain protecting groups are chemically cleaved from the peptide and the synthetic peptide from the resin support. Boc and Fmoc groups are the most widely used and commercially viable N-amino protecting groups for solid-phase peptide synthesis.

The protecting group of the alpha-amino acid can be removed by means of different acid reagents, for example, trifluoroacetic acid (TFA) or hydrochloric acid (HCl) dissolved in organic solvents at room temperature. After the removal of the protecting group of the alpha-amino acid, the remaining protected amino acids can be coupled step by step in the desired order. After the desired amino acid sequence has been assembled, the peptide is cleaved from the supporting resin by treatment with a reagent such as hydrogen fluoride (HF) which cleaves not only the peptide from the resin, but also the protecting groups of the lateral chains. When a chloromethylated resin or a hydroxymethylated resin is used, the treatment with HE leads to the formation of the terminal acid peptide in free form. When a BHA or p-Me-BHA resin is used, treatment with HF directly leads to the formation of the terminal amide peptide in free form.

The modification of the tryptophan residue to generate the modTrp for incorporation into a VIF peptide of the invention may be carried out either before, during or after the synthesis of the peptide. Modification of tryptophan to form a phenylthiocarbamate derivative can be carried out by reactions that are well known in the art. For example, a Boc-protected tryptophan moiety is reacted with phenylthiol (C₆H₅—SH), generally in the presence of HF. In one preferred method, a N-cyclohexyloxycarbonyl group is used as a protecting group in the synthesis of modTrp. (See, e.g., Nishiuchi et al., 1996.) An Fmoc group may be added to the N-terminal α-amino group of Trp by any of a number of methods well known in the art, for example, Fmoc may be added to the N-terminal amino group by methods routinely used in peptide synthesis.

As an alternative to chemical synthesis of the peptide, certain unmodified peptides can be produced recombinantly in a bacterial system by methods that are well known in the art. In such cases, modification of the tryptophan residue can then be carried out as described above for the chemically synthesized peptide. Such recombinant production of peptides may be desirable to provide large quantities of the peptides. Useful DNA sequences encoding the appropriate unmodified peptide are readily determined by one of ordinary skill in the art. The DNA encoding the unmodified peptide is preferably prepared using commercially available nucleic acid synthesis methods. Methods to construct expression systems for production of peptide in recombinant hosts are also generally known in the art. Expression can be effected in either procaryotic or eucaryotic hosts. Procaryotes most frequently are represented by various strains of E. coli. However, other microbial strains may also be used, such as bacilli, for example Bacillus subtilis, various species of Pseudomonas, or other bacterial strains. In such procaryotic systems, plasmid vectors which contain replication sites and control sequences derived from a species compatible with the host are used. Exemplary vectors for E. coli include pBR322, pUC 18 and derivatives thereof. Commonly used procaryotic control sequences, which contain promoters for transcription initiation, optionally with an operator, along with ribosome binding-site sequences, include the beta-lactamase (penicillinase) and lactose (lac) promoter systems, the tryptophan (trp) promoter system, and the lambda-derived PL promoter and N-gene ribosome binding site. However, any available promoter system compatible with procaryotes can be used.

Expression systems useful in eucaryotic hosts comprise promoters derived from appropriate eucaryotic genes. A class of promoters useful in yeast, for example, includes promoters for synthesis of glycolytic enzymes, e.g., those for 3-phosphoglycerate kinase. Other yeast promoters include those from the enolase gene or the Leu2 gene obtained from YEp13. Suitable mammalian promoters include the early and late promoters from SV40 or other viral promoters such as those derived from polyoma, adenovirus II, bovine papilloma virus or avian sarcoma viruses. An exemplary promoter for use in a plant expression system is the nopaline synthesis promoter. The expression systems are constructed and restriction, ligation and transformation into appropriate hosts is carried out using standard techniques appropriate to the type of cells being transformed. The cells containing the expression systems are cultured under conditions appropriate for production of the peptide, and the peptide is then recovered and purified.

The N-terminal α-amino group of such recombinantly-produced peptides may be carried out by any of a number of methods well known in the art, for example, Fmoc may be added to the N-terminal amino group by methods routinely used in peptide synthesis.

After a VIF amino acid or peptide of the present invention is produced by synthetic chemistry or through recombinant means, it is purified to substantial homogeneity by conventional protein purification techniques, for example reverse phase chromatography, HPLC, partition and/or ion exchange chromatography. The choice of appropriate matrices and buffers are well known in the art and are not described in detail herein.

V. Anti-Bacterial Amino Acid and Peptide Compositions for in vivo Delivery

The VIF amino acids and peptides of the present invention find utility in a method to treat or prevent infection of a subject by bacteria which produce a virulence factor, particularly Staphylococcus. One or more VIF amino acids or peptides, either alone or in a pharmaceutical composition with a pharmaceutically acceptable carrier, may be administered to the subject, as appropriate to the particular infection or may be integrated into a therapeutic or prosthetic device to lessen or prevent the occurrence of Staphylococcal infection associated with the use of the device.

In some cases, the VIF amino acids and peptides of the invention are administered together with an antibiotic. Antibiotics which have been used to treat Staphylococcal infection, include, but are not limited to, dicloxacillin; oxacillin; amoxicillin or ampicillin; cephalosporins, e.g., first generation cephalosporins (such as Cefadroxil or Duricef; Cephalexin or Keflex, and Cephradine or Velosef), second generation cephalosporins (such as Cefaclor or Ceclor, Cefuroxime Axtel or Ceftin, Cefprozil or Cefzil and Loracarbef or Lorabid) and third generation cephalosporins (such as Cefixime or Suprax, Cefpodoxime proxetil or Vantin, Ceftibuten or Cedax, Cefdinir or Omnicef and for intramuscular use Ceftriaxone or Rocephin); trimethoprim/sulfamethoxazole, vancomycin and methicillin.

Preferred antibiotics for combination therapy with one or more VIF amino acids or peptides of the invention include methicillin or vancomycin. The antibiotic may administered together with one or more VIF amino acids or peptides or separately. Such administration may take place at the same time or may be sequential.

The peptide compounds may be formulated into a composition in neutral or salt forms. Pharmaceutically acceptable nontoxic salts include the acid addition salts (formed by way of free amino groups) and those formed with inorganic acids such as, for example, hydrochloric, hydrobromic, sulfuric or phosphoric acids, or organic acids such as acetic, succinic, ascorbic, gluconic, benzoic, malic, fumaric, stearic, oxalic, tartaric, mandelic, and the like. Salts formed with the free carboxyl groups may be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Compositions for in vivo delivery take a variety of forms. These include, for example, solid, semi-solid, and liquid dosage forms, such as tablets, pills, powders, liquid solutions or suspensions, liposomes, and injectable and infusible solutions. The preferred form depends on the intended mode of administration and the therapeutic application. The compositions also preferably include conventional pharmaceutically acceptable carriers and adjuvants, as well known to those of skill in the art. See, e.g., Remington's Pharmaceutical Sciences, Mack Publishing Co., Easton, Pa., 18th Ed., 1990. Administration is generally carried out by topical, oral or parenteral (including subcutaneous, intramuscular, intravenous, and intradermal) routes. The therapeutic methods and agents of this invention can of course be used concomitantly or in combination with other methods and agents for treating a particular disease or disease condition, for example, conventional antibiotics (as further described above).

Liquid pharmaceutically administrable compositions can, for example, be prepared by dissolving, dispersing, etc., an effective amount of one or more VIF amino acids or peptides of the invention and optional pharmaceutical adjuvants in an excipient, such as, for example, water, saline, aqueous dextrose, glycerol, ethanol, olive oil, and other lipophilic solvents, and the like, to form a solution or suspension. If desired, the pharmaceutical composition to be administered may also contain minor amounts of nontoxic auxiliary substances, such as wetting or emulsifying agents, pH buffering agents, solubilizers, suspending agents, emulsifiers, buffers, thickening agents, colors viscosity regulators, preservatives stabilizers and osmolarity regulators and the like, for example, sodium acetate, sorbitan monolaurate, triethanolamine sodium acetate, triethanolamine oleate, etc. Actual methods of preparing such dosage forms are known and will be apparent to those skilled in this art; for example, see Remington's Pharmaceutical Sciences, supra.

Suitable examples of liquid carriers for parenteral administration of VIF amino acid or peptide preparations include water (partially containing additives, e.g., cellulose derivatives, preferably sodium carboxymethyl cellulose solution), alcohols (including monohydric alcohols and polyhydric alcohols, e.g., glycols) and their derivatives, and oils (e.g., fractionated coconut oil and arachis oil).

For parenteral administration of VIF amino acids or peptides, the carrier can also be an oily ester such as ethyl oleate and isopropyl myristate. Sterile carriers are useful in sterile liquid form compositions for parenteral administration.

The liquid carrier for pressurized compositions can be halogenated hydrocarbon or other pharmaceutically acceptable propellant. Such pressurized compositions may also be lipid encapsulated for delivery via inhalation. For administration by intranasal or intrabronchial inhalation or insufflation, VIF amino acids or peptides may be formulated in an aqueous or partially aqueous solution, which can then be utilized in the form of an aerosol.

For solid compositions, conventional nontoxic solid carriers can be used and include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 0.1-95% of active ingredient, preferably about 20%.

VIF amino acids and peptides may be administered topically as a solution, cream, powder, ointment, or lotion, by formulation with pharmaceutically acceptable vehicles containing the active compound. Lotions may be formulated with an aqueous or oily base and will, in general, also include one or more of the following: stabilizing agents, emulsifying agents, dispersing agents, suspending agents, thickening agents, coloring agents, perfumes, and the like. Powders may be formed with the aid of any suitable powder base, e.g., talc, lactose, starch, and the like. Drops may be formulated with an aqueous base or non-aqueous base also comprising one or more dispersing agents, suspending agents, solubilizing agents, and the like. Ointments and creams may, for example, be formulated with an aqueous or oily base with the addition of suitable thickening and/or gelling agents. Such bases may include water and/or an oil, such as liquid paraffin or a vegetable oil, such as peanut oil or castor oil. Thickening agents that may be used according to the nature of the base include soft paraffin, aluminum stearate, cetostearyl alcohol, propylene glycol, polyethylene glycols, woolfat, hydrogenated lanolin, beeswax, and the like.

The VIF amino acids and peptides may also be administered in liposome carriers. The use of liposome to facilitate cellular uptake is described, for example, in U.S. Pat. No. 4,897,355, U.S. Pat. No. 4,394,448, and U.S. Pat. No. 5,908,635. Numerous publications describe the formulation and preparation of liposomes.

The dosage requirements for treatment with VIF amino acids and peptides vary with the particular compositions employed, the route of administration, the severity of the symptoms presented, the form of VIF amino acid or peptide and the particular subject being treated. In general, a suitable effective dose of a VIF peptide and amino acid of the invention will generally be in the range of about 0.1 to 100 mg per kg of body weight per day and preferably in the range of between 0.1 to 10 mg per kg of body weight per day. It will be understood by those of skill in the art that the optimal dose is dependent upon a number of factors, particularly the compositions employed and the route of administration.

The desired dosage is preferably presented in one or more daily doses administered at appropriate intervals throughout the day. Preferably, the dosage is presented once per day in an amount that affords effective results without causing harmful or deleterious side effects (e.g., a pharmacologically effective amount). Such a concentration can be achieved by administration of a single unit dose, or by the administration of the same daily dose divided into convenient sub doses for administration at suitable intervals throughout the day.

VI. Methods of Treatment Using Anti-Bacterial Amino Acids or Peptides

The peptides of the present invention are typically administered to a host having or at risk of having a bacterial infection, e.g., a Staphylococcal infection.

In one aspect, the invention is directed to slowing or limiting bacterial infection, particularly Staphylococcal infection, in vivo in a human or animal subject, and/or a decrease in, or elimination of, detectable symptoms typically associated with infection by that particular bacteria.

It is appreciated that methods effective to deliver a VIF amino acid or peptide to the site of bacterial infection or to introduce a VIF amino acid or peptide into the bloodstream are contemplated.

The hosts are typically human patients. Animals may also be treated with the peptides of the present invention, including but not limited to animals of commercial or veterinary importance such as cows, sheep, pigs, horses, goats, dogs, cats and experimental animals such as rats, mice or guinea pigs.

In general, at least one VIF amino acid or peptide having a modified tryptophan residue is administered by topical, parenteral, or oral administration methods for prophylactic and/or therapeutic treatment.

Preferred VIF amino acids and peptides for in vivo are detailed in section IIA, above.

Topical administration may be used to deliver at least one VIF amino acid or peptide having a modified tryptophan residue to the subject by percutaneous passage of the VIF amino acid or peptide into the systemic circulation of the patient. The skin sites include anatomic regions for transdermally administering the compound, such as the forearm, abdomen, chest, back, buttock, and mastoidal area. The VIF amino acid or peptide is administered to the skin by placing on the skin either a topical formulation comprising the compound or a transdermal drug delivery device that administers the compound. In either embodiment, the delivery vehicle is designed, shaped, sized, and adapted for easy placement and comfortable retention on the skin.

A variety of transdermal drug delivery devices can be employed with the amino acids and peptides of this invention. For example, a simple adhesive patch comprising a backing material and an acrylate adhesive can be prepared. The amino acid or peptide and any penetration enhancer can be formulated into the adhesive casting solution. The adhesive casting solution can be cast directly onto the backing material or can be applied to the skin to form an adherent coating. See, e.g., U.S. Pat. Nos. 4,310,509; 4,560,555; and 4,542,012.

In other embodiments, the VIF amino acid or peptide of the invention is delivered using a liquid reservoir system drug delivery device. These systems typically comprise a backing material, a membrane, an acrylate based adhesive, and a release liner. The membrane is sealed to the backing to form a reservoir. The compound and any vehicles, enhancers, stabilizers, gelling agents, and the like are then incorporated into the reservoir. See, e.g., U.S. Pat. Nos. 4,597,961; 4,485,097; 4,608,249; 4,505,891; 3,843,480; 3,948,254; 3,948,262; 3,053,255; and 3,993,073.

Matrix patches comprising a backing, a drug/penetration enhancer matrix, a membrane, and an adhesive can also be employed to deliver a VIF amino acid or peptide of the invention transdermally. The matrix material typically will comprise a polyurethane foam. The amino acid or peptide, any enhancers, vehicles, stabilizers, and the like are combined with the foam precursors. The foam is allowed to cure to produce a tacky, elastomeric matrix which can be directly affixed to the backing material. See, e.g., U.S. Pat. Nos. 4,542,013; 4,460,562; 4,466,953; 4,482,534; and 4,533,540.

The VIF amino acids or peptides of the invention can also be delivered through mucosal membranes. Transmucosal (i.e., sublingual, buccal, and vaginal) drug delivery provides for an efficient entry of active substances to systemic circulation and reduces immediate metabolism by the liver and intestinal wall flora. Transmucosal drug dosage forms (e.g., tablet, suppository, ointment, pessary, membrane, and powder) are typically held in contact with the mucosal membrane and disintegrate and/or dissolve rapidly to allow immediate systemic absorption and may be used when a patient is unable to ingest a treatment composition orally.

For delivery to the buccal or sublingual membranes, typically an oral formulation, such as a lozenge, tablet, or capsule, will be used. The method of manufacture of these formulations is known in the art, including, but not limited to, the addition of the pharmacological agent to a pre-manufactured tablet; cold compression of an inert filler, a binder, and either a pharmacological agent or a substance containing the agent (as described in U.S. Pat. No. 4,806,356); and encapsulation. Another oral formulation is one that can be applied with an adhesive, such as the cellulose derivative hydroxypropyl cellulose, to the oral mucosa, for example as described in U.S. Pat. No. 4,940,587. Such a buccal adhesive formulation, when applied to the buccal mucosa, allows for controlled release of the pharmacological agent into the mouth and through the buccal mucosa.

Parenteral administration is generally characterized by injection, either subcutaneously, intramuscularly, or intravenously. Thus, this invention provides compositions for intravenous administration that comprise a solution of a VIF amino acid or peptide of the invention dissolved or suspended in an acceptable carrier. Injectables can be prepared in conventional forms, either as liquid solutions or suspensions, solid forms suitable for solution or suspension in liquid prior to injection, or as emulsions. Suitable excipients are, for example, water, buffered water, saline, dextrose, glycerol, ethanol, or the like. These compositions will be sterilized by conventional, well known sterilization techniques, such as sterile filtration. The resulting solutions can be packaged for use as is or lyophilized, the lyophilized preparation being combined with a sterile solution prior to administration. In addition, if desired, the pharmaceutical compositions to be administered may also contain minor amounts of non-toxic auxiliary substances, such as wetting or emulsifying agents, pH buffering agents and the like, such as for example, sodium acetate, sorbitan monolaurate, triethanolamine oleate, etc.

Parenteral administration employs the implantation of a slow-release or sustained-release system, such that a constant level of dosage is maintained. See, e.g., U.S. Pat. No. 3,710,795.

VII. Antibacterial Devices

In a separate embodiment, the present invention provides a method for preventing or reducing the occurrence of a bacterial infection, particularly a Staphylococcal infection, associated with use of a medical device, for example, a therapeutic or prosthetic device.

Indwelling medical devices including vascular catheters are used with increasing frequency in the treatment of hospitalized patients by providing venous access. The benefit derived from these catheters as well as other types of medical devices such as peritoneal catheters, cardiovascular devices, orthopedic implants and other prosthetic devices is often offset by infectious complications. The most common organisms causing these infectious complications are Staphylococcus epidermidis and Staphylococcus aureus.

The method of the present invention provides a means to prevent or reduce the occurrence of Staphylococcal infections commonly associated with the use of such medical devices. The method comprises integrating a VIF amino acid or peptide of the invention into a medical device. The VIF amino acid or peptide can be integrated into the device in any manner appropriate to the condition under treatment, many of which are known in the art. For example, the device can be coated with any appropriate formulation of a VIF amino acid or peptide, or can be contacted with a solution of VIF amino acid or peptide under conditions in which the amino acid or peptide adheres to, or is absorbed into, the device. The device can also be fabricated from materials into which the amino acid or peptide has been incorporated. Some methods for integrating the amino acid or peptide into the device can be found in U.S. Pat. Nos. 5,902,238, 5,756,145, 5,853,745 and 5,788,979.

Particular devices especially suited for integration of the amino acid or peptides of the invention include peripherally insertable central venous catheters, dialysis catheters, long term tunneled central venous catheters, peripheral venous catheters, short-term central venous catheters, arterial catheters, pulmonary artery Swan-Ganz catheters, urinary catheters, long term urinary devices, tissue bonding urinary devices, penile prostheses, vascular grafts, vascular catheter ports, wound drain tubes, hydrocephalus shunts, peritoneal catheters, pacemaker capsules, artificial urinary sphincters, small or temporary joint replacements, urinary dilators, heart valves and the like.

The following examples illustrate but are not intended in any way to limit the invention.

### EXAMPLE 1

Discovery of a Virulence Inhibitory Factor (VIF)

Synthetic PEP (YSPWTNF, SEQ ID NO: 1), obtained from a commercial protein synthesis laboratory (Penta Biotech, Foster city, CA) was tested for its effect on α-toxin production by a clinical isolate of S. aureus (“wild Staphylococcus” or “WS”), previously determined to produce high levels of β-toxin. WS cells were initiated in culture at 3×10⁸/ml and cultured in the presence or absence of test peptide for 2-3 hrs. After 2-3 hours, the culture supernatant was assayed for hemolytic activity on rabbit erythrocytes according to a previously published procedure (Bernheimer, 1988), which is highly sensitive to α-toxin, but may also detect other hemolytic toxins, if present.

In contrast to previously observed activity, purified, synthetic PEP (SEQ ID NO:1) had similar activity to a scrambled peptide control (which contains the same amino acids as PEP), in α-toxin production and RNA III assays.

Conventional Merrifield solid-phase peptide synthesis peptide was carried to synthesize a peptide having the sequence TyrSerProTrpThrAsnPhe (Merrifield, 1963). The reaction product was then subjected to fractionation on a C4 reverse phase HPLC column and each fraction tested for inhibition of α-toxin production. Strong inhibitory activity was correlated with a peak of material which eluted at 36 minutes, although the major component was a peak of material which appeared to contain a single component and eluted at 41 minutes. Amino acid sequencing of the 41 min fraction yielded the sequence of TyrSerProTrpThrAsnPhe (the sequence of the PEP peptide).

The peak of material eluting at 36 minutes, appeared to consist of two closely eluting components, which were further separated on a C18 column. The later eluting peak contained the material which was active in the α-toxin assay. The purified active material was subjected to amino acid sequencing and the sequence was determined to be TyrSerProTrpThrAsnPhe (identical to that of the PEP peptide). However, the molecular weight as determined by mass spectrophotometry (MS) was 1273, which is higher than the predicted mass of 913 for PEP (TyrSerProTrpThrAsnPhe). The difference in mass suggested that the tryptophan residue was modified at the indole side chain, consistent with modification of Trp to form a phenylthiocarbamate derivative. While the mechanism is not part of the present invention, formation of the S-phenyl thiocarbamate derivative of Trp is consistent with the solid phase peptide synthesis method. In the last step of the solid phase synthesis of the peptide, under strongly acidic conditions, thioanisole can be converted to phenylthiol which can further react with the carbonyl group of Boc protected Trp to form a phenylthiocarbamate derivative. The active peptide containing modTrp is designated “VIF-1” for virulence inhibitory factor 1.

VIF-1 Inhibits Production of Virulence Factors by all S. aureus Groups Tested and S. warneri.

VIF-1 (SEQ ID NO: 2) was evaluated for its effect on in vitro virulence factor production by testing with strains of S. aureus representing the four different interference groups (Ji et al. 1997). The results demonstrate that VIF-1 dose-dependently inhibited α-toxin production by all four groups of S. aureus with IC₅₀ values ranging from 0.06 to 0.15 μg/ml (Table 1). Given that the concentrations of VIF-1 peptide were based on testing the synthetic peptide prior to column purification, the actual IC₅₀ values for pure VIF-1 will be lower, probably around 20% of the observed values. The scrambled control peptide was inactive. The synthetic peptides did not inhibit bacterial growth in 24 hr WS cultures (data not shown).

Although S. warneri is not a frequent pathogen, it has been reported to cause infections, and some isolates produce exoproteins such as α- and δ-toxins (Lambe et al., 1990). Hemolytic activity secreted by S. warneri was detected, although the amount was smaller than that produced by S. aureus. However, VIF-1 potently inhibited hemolysin production by S. warneri. This result is significant, since none of the other modified AIPs previously described (Mayville et al., 1999; Otto et al., 1999) could inhibit agr activation and production of virulence factors by the autologous strain from which the modified AIP was derived.

TABLE 1
IC₅₀ Values for Inhibition of Hemolysin Production by VIF-1

| Staphylococci | Group | IC₅₀ (μg/ml) |
|---|---|---|
| Wild | | 0.10 |
| RN6734 | I | 0.06 |
| 27217 | II | 0.10 |
| RN3984 | III | 0.15 |
| RN4850 | IV | 0.10 |
| RN833 | S. warneri | 0.10 |

The effects of VIF-1 on toxic shock syndrome toxin-1 (TSST-1), under the control of agr, and an important virulence factor in many cases of S. aureus infection, were evaluated. The results show that VIF-1 suppressed release of TSST-1 by all four groups of S. aureus at concentrations as low as 0.2 μg/ml (Table 2). S. warneri was not tested in this experiment, since it does not produce detectable amounts of TSST-1.

The amount of TSST-1 in the supernatants of cells cultured for four hours with or without VIF-1 was measured in an ELISA. VIF-1 was added to the bacterial culture to a concentration of 0.04 μg/ml, 0.2 μg/ml or 1.0 μg/ml. Rabbit polyclonal antibodies directed against TSST-1 as well as purified TSST-1 were obtained from Toxin Technology, Inc. (Sarasota, Fla.). Microtiter plates were coated with 0.05 ml of rabbit antibodies directed against TSST-1 at 4.0 μg/ml in PBS. Plates were incubated overnight at room temperature and washed 3 times with water. Wells were then filled with blocking buffer (0.05% Tween 20) and incubated for 30 min. at room temperature, followed by rinsing 3 times with water. The test sample (0.05 ml) was added and incubated 2 hr. at room temperature. A standard curve was prepared by adding known amounts of purified TSST-1 to quantitate the Staphylococcal supernatants. The rabbit anti-TSST-1 antibodies were conjugated to biotin using a commercially available kit (Pierce) and added to the plate at 1.0 μg/ml. diluted in blocking buffer. After incubation 2 hr, at room temperature, the plate was rinsed 3 times with water, then 0.05 ml of streptavidin conjugated to alkaline phosphatase was added at 200 μg/ml., incubated 2 hr., and rinsed 3 times. Then 0.075 ml of p-nitrophenyl phosphate at 0.2 μg/ml. was added and after a 1 hour incubation at room temperature, the OD was read at 405 nm in a plate reader. The results are shown in Table 2.

TABLE 2
Inhibition of TSST-1 Production by VIF-1.

| | TSST-1 (ng/ml) | | | |
|---|---|---|---|---|
| | | + VIF-1 | + VIF-1 | + VIF-1 |
| S. aureus strain | no VIF-1 | (1.0 μg/ml) | (0.2 μg/ml) | (0.04 μg/ml) |
| WS | 15.2 | 0.1 | 0.6 | 14.5 |
| RN6734 | 15.4 | 0.6 | 1.6 | 16.3 |
| 27217 | 15.0 | 0.8 | 0.8 | 16.0 |
| RN3984 | 14.0 | 0.4 | 0.6 | 14.4 |
| RN4850 | 19.5 | 1.2 | 1.4 | 27.0 |

### EXAMPLE 2

In vitro Anti-Staphylococcal Activity of VIF Amino Acids and Peptides

The anti-Staphylococcal activity of a number of commercially available amino acids and peptides (Chem-Impex International, IL) was evaluated by an in vitro RNAIII induction assay and in an assay for α-toxin virulence factor production, as described above. In addition, a number of modified amino acid were synthesized using techniques routinely employed by those of skill in the art. Exemplary compounds are presented in Table 3.

TABLE 3
Synthetic Amino Acid And Peptides Tested
For Anti-Bacterial Activity.

| Description | Reference # |
|---|---|
| Boc-L-Trp(CO-S-C6H5-OH | 1 |
| Fmoc-D-2-naphthylalanine | 2 |
| Fmoc-L-2-naphthylalanine | 3 |
| Fmoc-2-aminobenzoic acid | 4 |
| Fmoc-4-aminobenzoic acid | 5 |
| Fmoc-N-beta-Boc-L-diaminopropionic acid | 6 |
| Fmoc-N-beta-Fmoc-L-diaminopropionic acid | 7 |
| Fmoc-N-beta-Z-L-diaminopropionic acid | 8 |
| Fmoc-N-beta-alloc-L-diaminopropionic acid | 9 |
| Fmoc-L-indoline-2-carboxylic acid | 10 |
| Fmoc-1,2,3,4-D-tetrahydroisoquinoline-3-carboxylic acid | 11 |
| Fmoc-1,2,3,4-L-tetrahydroisoquinoline-3-carboxylic acid | 12 |
| Fmoc-D-3-pyridylalanine | 13 |
| Fmoc-2-aminoindane-2-carboxylic acid | 14 |
| Fmoc-(D,L)-2-aminotetraline-2-carboxylic acid | 15 |
| Fmoc-D-tetrahydroisoquinoline-1-carboxylic acid | 16 |
| (R,S)-Fmoc-1,3-dihydro-2H-isoindole carboxylic acid | 17 |
| Fmoc-D-1,2,3,4-tetrahydronorharman-3-carboxylic acid | 18 |
| Fmoc-4-phenyl-piperidine-4-carboxylic acid | 19 |
| Fmoc-4-phenyl-pyrrolidine-2-carboxylic acid | 20 |
| (R,S)-Fmoc-1-aminoindane-1-carboxylic acid | 21 |
| (R,S)-N-Fmoc-N′-Boc-imidazolidine-2-carboxylic acid | 22 |
| 4-(Fmoc-2-aminoethyl)-6-dibenzofuranpropionic acid | 23 |
| Fmoc-3-amino-3-(1-naphthyl)-propionic acid | 24 |
| Fmoc-L-Trp(CO-S-C6H5)-OH | 25 |
| Peptide H-YSPW*TNF-OH | 26 |
| Peptide H-YSPW*TNF-OH (Side-product MW = M + 108) | 27 |
| Fmoc-L-Cys(Acm)-OH | 28 |
| Fmoc-L-His(Trt)-OH | 29 |
| Fmoc-L-Lys(Boc)-OH | 30 |
| Fmoc-L-Met-OH | 31 |
| Fmoc-L-Phe-OH | 32 |
| Fmoc-L-Ser(tBu)-OH | 33 |
| Fmoc-L-Trp-OH | 34 |
| Fmoc-L-Trp(Boc)-OH | 35 |
| Fmoc-L-Tyr-OH | 36 |
| Fmoc-L-Tyr(tBu)-OH | 37 |
| Fmoc-L-Val-OH | 38 |
| Ac-L-Trp-Oet | 39 |
| Ac-L-Trp-OH | 40 |
| Boc-L-Phe | 41 |
| Fmoc-L-His | 42 |
| Fmoc-L-His(Boc)-OH | 43 |
| H-L-Phe-Ome-HCl | 44 |
| H-L-Trp(Boc)-OH | 45 |
| H-L-Trp-OH | 46 |
| H-L-Tyr(tBu)-OMe, HCl | 47 |
| H-L-Val-OtBu, HCl | 48 |
| Boc-N-beta-Fmoc-D-diamimopropionic-acid | 49 |
| Boc-N-beta-Fmoc-L-diaminopropionic-acid | 50 |
| Fmoc-OSu | 51 |
| Fmoc-L-Ala-OH | 52 |
| Fmoc-L-Arg(Pmc)-OH | 53 |
| Fmoc-L-Arg(Tos)-OH | 54 |
| Fmoc-L-Arg(Pbf)-OH | 55 |
| Fmoc-L-Asn(Trt)-OH | 56 |
| Fmoc-L-Asp(OAll)-OH | 57 |
| Fmoc-L-Asp(OBzl)-OH | 58 |
| Fmoc-L-Asp(OtBu)-OH | 59 |
| Fmoc-L-Asp(OcHx)-OH | 60 |
| Fmoc-L-Cys(Allocam)-OH | 61 |
| Fmoc-L-Cys(tBu)-OH | 62 |
| Fmoc-L-Cys(4McBzl)-OH | 63 |
| Fmoc-L-Cys(Trt)-OH | 64 |
| Fmoc-L-Gln(Trt)-OH | 65 |
| Fmoc-L-Ile-OH | 66 |
| Fmoc-L-Leu-OH | 67 |
| Fmoc-L-Lys(Alloc)-OH | 68 |
| Fmoc-L-Lys(Z)-OH | 69 |
| Fmoc-L-Lys(2-Cl-Z)-OH | 70 |
| Fmoc-L-Lys(Dnp)-OH | 71 |
| Fmoc-L-Lys(Fmoc)-OH | 72 |
| Fmoc-4-bromo-L-phenylalanine | 73 |
| Fmoc-4-methyl-L-phenylalanine | 74 |
| Fmoc-L-Pro-OH | 75 |
| Fmoc-L-Scr(Bzl)-OH | 76 |
| Fmoc-L-Thr(Bzl)-OH | 77 |
| Fmoc-D-Trp(Boc)-OH | 78 |
| Fmoc-L-Tyr(All)-OH | 79 |
| Fmoc-L-Tyr(2-Br-Z)-Oh | 80 |
| Fmoc-L-Tyr(2,6-di-Cl-Bzl)-OH | 81 |
| Fmoc-beta-cyclohexyl-L-alanine | 82 |
| Fmoc-beta-(2-thienyl)-L-alanine | 83 |
| Fmoc-L-1-naphthylalanine | 84 |
| Fmoc-(4-aminomethyl)-benzoic acid | 85 |
| Fmoc-1-amino-1-cyclohexane carboxylic acid | 86 |
| Fmoc-2-aminoisobutyric acid | 87 |
| Fmoc-(3S,4S)-4-amino-3-hydroxy-5-phenyl-Pentanoic acid | 88 |
| Fmoc-L-t-butylglycine | 89 |
| Fmoc-D-phenylglycine | 90 |
| Fmoc-L-phenylglycine | 91 |
| Fmoc-4-chloro-L-D-phenylalanine | 92 |
| Fmoc-3,4-dichloro-L-phenylalanine | 93 |
| Fmoc-4-fluoro-L-phenylalanine | 94 |
| Fmoc-4-nitro-L-phenylalanine | 96 |
| Fmoc-3-nitro-L-tyrosine | 97 |
| Fmoc-L-3-pyridylalanine | 98 |
| Fmoc-isonipecotic acid | 99 |
| Fmoc-(4-carboxymethyl)-piperidine | 100 |
| Fmoc-L-tetrahydroisoquinoline-1-carboxylic acid | 101 |
| Fmoc-(4-piperidyl)-L-proline | 102 |
| Fmoc-tranexamic acid | 103 |
| Racemic Fmoc-trans-3-phenyl-azetidine-2-carboxylic acid | 104 |
| Fmoc-L-citrulline | 105 |
| Fmoc-4-carboxymethyl-piperazine | 106 |
| (R,S)-Fmoc-2-carboxymorpholine | 107 |
| Fmoc-4-(2-aminoethyl)-1-carboxymethyl-piperazine | 108 |
| dihydrochloride | |
| (2S,4S)-Fmoc-4-amino-1-Boc-pyrrolidine-2-carboxylic acid | 109 |
| (2S,4S)-Fmoc-4-amino-1-benxoyl-pyrrolidine-2-carboxylic | 110 |
| acid | |
| Fmoc-3-amino-1-carboxymethyl-pyridin-2-one | 111 |
| Fmoc-L-1,2,3,4-tetrahydronorharman-3-carboxylic acid | 112 |
| (2S,5R)-Fmoc-5-phenyl-pyrrolidine-2-carboxylic acid | 113 |
| Fmoc-nipecotic acid | 114 |
| Fmoc-piperazine hydrochloride | 115 |
| Fmoc-L-4-pyridylalanine | 116 |
| Fmoc-N-(cyclohexyl)-glycine | 117 |
| Fmoc-L-pipecolic acid | 118 |
| Fmoc-3-axetidine carboxylic acid | 119 |
| Fmoc-L-azetidine-2-carboxylic acid | 120 |
| Fmoc-4-amino-1-carboxymethyl-piperidine | 121 |
| Fmoc-4-aminomethyl-phenylacetic acie | 122 |
| Fmoc-L-3,4-dimethoxyphenylalanine | 123 |
| Fmoc-L-styrylalanine | 124 |
| Fmoc-4-hydrazinobenzoic acid | 125 |
| (R)-Fmoc-4-amino-5-phenyl-pentanoic acid | 126 |
| (R,S)-Fmoc-3-amino-3-(4-bromophenyl)-propionic acid | 127 |
| (R,S)-Fmoc-3-amino-3-(1-naphthyl)-propionic acid | 128 |
| (R,S)-Fmoc-3-amino-N-1-carboxymethyl-2-oxo-5-phenyl-1, | 129 |
| 4-benzodiazepine | |
| (3S)-Fmoc-3-amino-1-carboxymethy-caprolatame | 130 |
| Fmoc-3-(2-aminoethyl)-1-carboxy-methyl-quinazoline-2, | 131 |
| 4-dione | |
| Fmoc-BTD | 132 |
| (2S,5S)-Fmoc-5-amino-1,2,4,5,6,7-hexahydro-azepino [3,2, | 133 |
| 1-hi] indole-4-one-2-carboxylic acid | |
| (2S,6S,9S)-Fmoc-6-amino-2-carboxymethyl-3,8- | 134 |
| diazabicyclo-[4,3,0]-nonane-1,4-carboxylic acid | |
| Fmoc-4-piperidone | 135 |
| Fmoc-3-carboxymethyl-1-phenyl-1,3,8-triazaspiro[4, | 136 |
| 5]decan-4-one | |
| Fmoc-4-(3-carboxymethyl-2-keto-1-benzimidazolyl)- | 137 |
| piperidine | |
| Emoc-Aixa | 138 |
| 6-(N-Fmoc-piperazin1-yl-4(3H)-quinazolinone-3-acetic acid | 139 |
| Fmoc-4-carboxymethyl-1,2,3,4-tetrahydroquinoxalin-3-one | 140 |
| (Fmoc-4-amino)-piperidine hydrochloride | 141 |
| N-alpha-Z-N-beta-Fmoc-L-diaminopropionic acid | 142 |
| Boc-L-Trp(CHO) | 143 |

The RNAIII induction assay was carried out using the RNAIII: β-lactamase fusion construct (rnaiii:blaZ), and the β-lactamase activity in the supernatant was measured colorimetrically. The results presented in Tables 4, 5 and 6 indicate that VIF-1 (SEQ ID NO:2) and a number of synthetic amino acids have significant anti-Staphylococcal activity. In contrast, a number of the synthetic amino acids listed in table 3, were not active in an α-hemolysin assay with an IC₅₀ of >50 μg/ml.

TABLE 4
Inhibition of RNA III Activation and α-Hemolysin
Production by Modified Amino Acids

| | | RNA III | α-Hemolysin | TSST-1 |
|---|---|---|---|---|
| Ref. # | Compound | μg/ml (n) | (μg/ml) (n) | (μg/ml) |
| N/A | VIF-1 | 1.5 | 1.1 | |
| 35 | Fmoc-Trp(Boc)-OH (FTB) | 1.4 | 1.2 (13) | 5.0 |
| 34 | Fmoc-Trp | >2.5 | 9.0 | NT |
| 45 | H-Trp(Boc)-OH | >10.0 | >50 | NT |
| 42 | Fmoc-His-OH | NA | 47 | NT |
| 43 | Fmoc-His(Boc)-OH | NA | >50 | NT |
| 32 | Emoc-Phe | >2.5 | 1.3 | NT |
| 38 | Emoc-Val | >2.5 | 1.5 | NT |
| 4 | FMoc-2-aminobenzoic acid | 2.9 (3) | 0.9 (5) | 2.0 |
| 13 | Fmoc-D-3-pyridylalanine | NT | >50 | NT |
| 16 | Fmoc-D-tetrahydroisoquinoline-1-carboxylic | 3.5 (3) | 1.3 (3) | 4.0 |
| | acid | | | |
| 18 | Fmoc-L-1,2,3,4-tetrahydronorharman-3- | NT | 2.0 (5) | NT |
| | carboxylic acid | | | |
| 29 | Fmoc-L-His(Trt)-OH | 3.7 (3) | 2.7 (3) | 4.0 |
| 46 | H-L-Trp-OH | >10.0 (3) | NT | >10.0 |
| 52 | Fmoc-L-Ala-OH | NT | 6.5 | NT |
| 67 | Fmoc-L-Leu-OH | NT | 2.1 (4) | NT |
| 69 | FMoc-2-L-Lys(Z)-OH | 4.7 (3) | 2.8 (4) | NT |
| 73 | Fmoc-4-bromo-L-phenylalanine | NT | 1.7 (5) | NT |
| 74 | Fmoc-4-methyl-L-phenylalanine | NT | 2.3 (6) | NT |
| 78 | Fmoc-D-Trp(Boc)-OH | NT | 1.3 (5) | NT |
| 82 | Fmoc-beta-cyclohexyl-L-alanine | NT | 4.0 | NT |
| 83 | Fmoc-beta-(2-thienyl)-L-alanine | NT | 25 | NT |
| 86 | Fmoc-1-amine-cyclohexane carboxylic acid | 3.0 (3) | 2.1 (3) | 10.0 |
| 92 | Fmoc-4-chloro-L-phenylalanine | NT | 1.6 (5) | NT |
| 104 | Racemic Fmoc-trans-3-phenyl-azetidine-2- | NT | 3.9 | NT |
| | carboxylic acid | | | |
| 113 | Fmoc-5-phenyl-pyrrolidine-2-carboxylic acid | 3.0 (1) | 1.7 (5) | 6.0 |
| 120 | Fmoc-L-azetidine-2-carboxylic acid | NT | 12.0 | NT |
| 124 | FMoc-2-L-styrylalanine | 2.9 (1) | 2.0 (4) | 10.0 |
| 126 | (R)-Fmoc-4-amino-5-phenyl-pentanoic acid | 9.0 (2) | 1.7 (4) | NT |
| 127 | (R,S)-Fmoc-3-amino-1-cyclohexane carboxylic | NT | 1.4 (3) | NT |
| | acid | | | |

The relative activity of VIF-1 (SEQ ID NO:2), Fmoc-Trp(Boc)-OH, and a number of selected synthetic amino acids previously demonstrated to exhibit anti-bacterial activity (reference numbers 4, 86, 16, 113 and 124) and a synthetic amino acid demonstrated to lack anti-bacterial activity (reference number 98), on in vitro virulence factor production was evaluated by testing α-toxin production using strains of S. aureus representing the four different interference groups (Ji et al. 1997), S. warnerii, S. epidernidis and S. haemolyticus.

The results presented in Table 5 demonstrate that VIF-1, Fmoc-Trp(Boc)-OH (FTB), Fmoc-2-aminobenzoic acid (reference #4); Fmoc-1-amine-cyclohexane carboxylic acid (reference #86); Fmoc-D-tetrahydroisoquinoline-1-carboxylic acid (reference #16); Fmoc-5phrnly-pyrrolidine-2-carboxylic acid (reference #113); and FMoc-2-L-styrylalanine (reference #124) inhibited α-toxin production by all four groups of S. aureus and S. warnerii and that Fmoc-L-pyridylalanine (reference #98) was not inhibitory.

TABLE 5
Effect Of Anti-Bacterial Amino Acid And VIF-1 on α-Toxin Production

| | Staph | IC₅₀ (μg/ml) | | | | | | | |
|---|---|---|---|---|---|---|---|---|---|
| Staph Strain | Group | VIF | FTB | #4¹ | #86² | #16³ | #113⁴ | #124⁵ | #98⁶ |
| Wild S. aureus | | 2.5 | 0.8 | 0.9 | 1.0 | 1.1 | 0.8 | 0.5 | >5 |
| RN6734 | I | 0.6 | 1.5 | 0.4 | 1.5 | 1.2 | 1.6 | 0.4 | >5 |
| RN27217 | II | 0.6 | 1.8 | 1.1 | 1.4 | 1.5 | 1.9 | 0.5 | >5 |
| RN3984 | III | 0.6 | 0.8 | 0.7 | 0.9 | 0.8 | 0.9 | 0.6 | 1.0 |
| RN4850 | IV | 2.4 | 0.8 | 0.2 | 0.8 | 0.8 | 0.9 | 0.2 | 4.8 |
| S. warnerii | | 0.1 | 0.6 | 0.6 | 0.6 | 0.6 | 0.6 | 0.7 | 4.5 |
| S. epidermidis (51625) | | | 0.5 | 0.5 | 0.4 | 0.5 | 0.5 | 0.6 | 3.0 |
| S. haemolyticus | | | 0.6 | 0.7 | 0.6 | 0.6 | 0.7 | 0.5 | 3.5 |

The relative activity of VIF-1 (SEQ ID NO:2), Fmoc-Trp(Boc)-OH (FTB) and a number of selected synthetic amino acids was further evaluated by an assay of Staphylococcus growth. Aliquots of Staphylococcus were pipetted into wells of a flat bottom microtiter plate along with various dilutions of the compound to be tested. The plates were incubated overnight, and examined the following day for growth, indicated by turbidity. The lowest dilution of compound that inhibited the growth of Staphylococcus, yielding a non-turbid well, was deemed the Minimal Inhibitory Concentration (MIC). Vancomycin was used as a positive control for growth ¹ Reference #4 is Fmoc-2-aminobenzoic acid ² Reference #86 is Fmoc-1-amine-cyclohexane carboxylic acid ³ Reference #16 is Fmoc-D-tetrahydroisoquinoline-1-carboxylic acid ⁴ Reference #113 is Fmoc-5-phenyl-pyrrolidine-2-carboxylic acid ⁵ Reference #124 is FMoc-2-L-styrylalanine ⁶ Reference #98 is FMoc-L-pyridylalanine

inhibition. (See Table 6). The results shown in Table 6 indicate that the compounds are not growth inhibitors.

TABLE 6
Inhibition Of Staph Growth By VIF Analogs.

| | | | MIC⁷ |
|---|---|---|---|
| Compound | Ref. # | n | μg/ml +/− sem |
| VIF | N/A | 1 | 25 |
| FTB | | 3 | 12.5 +/− 0 |
| Fmoc-2-aminobenzoic acid | 4 | 3 | 7.2 +/− 1.6 |
| Fmoc-1-amino-1-cyclohexane | 86 | 3 | 50 +/− 0 |
| carboxylic acid | | | |
| Fmoc-D-tetrahydroisoquinoline-1- | 16 | 2 | 25 |
| carboxylic acid | | | |
| (2S,5R)-Fmoc-5-phenyl-pyrrolidine-2- | 113 | 2 | 25 |
| carboxylic acid | | | |
| (R)-Fmoc-4-amino-5-phenyl-pentanoic | 126 | 1 | NA |
| acid | | | |
| Fmoc-L-His(Trt)-OH | 29 | 1 | NA |
| Fmoc-L-styrylalanine | 124 | 2 | 25 |
| Fmoc-L-Lys(Z)-OH | 69 | 1 | 50 |
| Fmoc-L-3-pyridylalanine | 98 | 1 | 50 |
| H-L-Trp-OH | 46 | 1 | 50 |
| Fmoc-L-citrulline | 105 | 1 | 50 |

### EXAMPLE 3

In vivo Anti-Staphylococcal Activity of VIF Amino Acids and Peptides

The anti-Staphylococcal activity of VIF amino acids and a VIF peptide (VIF-1; SEQ ID NO:2) based on in vitro assays, was further evaluated in an in vivo mouse model for Staphylococcal infection.

The assay was carried out by injecting a fixed amount of a S. aureus culture, typically containing from about 5×10⁹ cells from an early exponential growth stage culture, by intraperitoneal (IP) injection into mice, based on the method described by Thakker et al., 1998.

In one study, the S. aureus was mixed with VIF-1 or FTB, prior to IP injection. The results shown in Table 7 indicate that 3 or 15 mg/kg of VIF-1 and 1, 2 or 5 mg/kg of FTB were effective to significantly increase the number of mice that survived at 24 hours post-injection.

TABLE 7
Activity in Mouse Model With Staph Mixed With
Drug Prior to IP Iniection (24 h).

| Treatment | n | % Survival at 24 hr. |
|---|---|---|
| Vehicle | 14 | 0 |
| VIF 1 mg/kg | 3 | 67 |
| VIF 3 mg/kg | 6 | 83 |
| VIF 15 mg/kg | 6 | 83 |
| Scrambled peptide 15 mg/kg | 3 | 33 |
| FTB 0.2 mg/kg | 2 | 0 |
| FTB 1 mg/kg | 6 | 83 |
| FTB 2 mg/kg | 3 | 67 |
| FTB 5 mg/kg | 6 | 83 |
| Trp 1 mg/kg | 2 | 0 |
| Trp 5 mg/kg | 3 | 33 |

⁷MIC refers to minimal inhibitory concentration

B. In vivo Anti-Staphylococcal Activity of VIF Amino Acids and Peptides in Combination With Antibiotic Therapy

In another approach, S. aureus was injected IP 20 minutes prior to IP injection of VIF-1 or FTB. The results shown in Table 6 indicate that 20 or 15 mg/kg of VIF-1 and 10 mg/kg of FTB were effective to significantly increase the number of mice that survived at 24 hours post-injection relative to the vehicle control.

The commonly used anti-Staphylococcal antibiotics, methicillin and vancomycin, were tested in this model. A 1 mg/kg dose of methicillin and a 0.5 mg/kg dose of vancomycin resulted in 17% survival of mice at 24 hours. However, when 1 mg/kg of methicillin and 0.5 mg/kg of vancomycin was administered together with a dose of VIF (6 mg/kg) or FTB (2 mg/kg) (which when given alone resulted in 13% and 25% survival of mice at 24 hours), enhanced survival was observed, suggesting a synergistic effect based on the combination of VIF or FTB with methicillin and vancomycin (Table 8).

TABLE 8
Cumulative Results of Mouse Model With Staph Injected
20 Min. Prior to Drug Injection (IP)

| Treatment | n | % Survival at 24 hr. |
|---|---|---|
| Vehicle | 14 | 0 |
| VIF 20 mg/kg | 6 | 67 |
| VIF 15 mg/kg | 2 | 50 |
| VIF 6 mg/kg | 8 | 13 |
| FTB 10 mg/kg | 7 | 71 |
| FTB 2 mg/kg | 4 | 25 |
| methicillin 100 mg/kg | 2 | 100 |
| methicillin 10 mg/kg | 2 | 50 |
| methicillin 1 mg/kg | 6 | 17 |
| vancomycin 50 mg/kg | 2 | 100 |
| vancomycin 5 mg/kg | 2 | 50 |
| vancomycin 0.5 mg/kg | 6 | 17 |
| VIF 6 mg/kg + meth. 1 mg/kg | 6 | 50 |
| VIF 6 mg/kg + vanc. 0.5 mg/kg | 6 | 100 |
| FTB 2 mg/kg + meth. 1 mg/kg | 6 | 83 |
| FTB 2 mg/kg + vanc. 0.5 mg/kg | 6 | 83 |

Table Of Sequences.

| | SEQ | FIG. |
|---|---|---|
| DESCRIPTION | ID NO | NO. |
| TyrSerProTrpThrAsnPhe or YSPWTNF (“PEP”) | 1 | 1A |
| Fmoc-TyrSerPro-(modifiedTrp)-ThrAsnPhe, wherein | 2 | 1B |
| modifiedTRP is an S-phenylthiocarbamate derivative | | |
| of tryptophan (VIF-1) | | |
| TyrSerPro-(modifiedTrp)-ThrAsnPhe, wherein | 3 | |
| modifiedTrp is an S-phenylthiocarbamate derivative of | | |
| tryptophan (VIF-1 without Fmoc) | | |
| TyrSerPro-(modifiedTrp)-ThrAsnPhe, modifiedTrp has a | 4 | 1B |
| BOC group linked to the ring nitrogen | | |
| Fmoc-TyrSerPro(modifiedTrp), wherein the modifiedTrp | 5 | |
| is a phenylthiocarbamate derivative of tryptophan | | |
| Fmoc-TyrSerPro(modifiedTrp), wherein the modifiedTrp | 6 | |
| has a BOC group linked to the ring nitrogen | | |

All publications, patents and patent applications are herein expressly incorporated by reference in their entirety to the same extent as if each individual publication, patent or patent application was specifically and individually indicated to be incorporated by reference in its entirety.

It is claimed:

1. An isolated synthetic peptide inhibitory of Staphylococcal virulence selected from the group consisting of Fmoc-TyrSerPro(modifiedTrp)ThrAsnPhe, wherein the modifiedTrp is an S-phenylthiocarbamate derivative of tryptophan (SEQ ID NO:2); TyrSerPro(modifiedTrp)ThrAsnPhe, wherein the modifiedTrp is an S-phenylthiocarbamate derivative of tryptophan (SEQ ID NO:3); Fmoc-TyrSerPro (modifiedTrp) ThrAsnPhe, wherein the modifiedTrp has a BOC group linked to the ring nitrogen (SEQ ID NO:4); Fmoc-TyrSerPro(modifiedTrp), wherein the modifiedTrp is a phenylthiocarbamate derivative of tryptophan (SEQ ID NO:5); Fmoc-TyrSerPro(modifiedTrp), wherein the modifiedTrp has a BOC group linked to the ring nitrogen (SEQ ID NO:6).

2. The peptide of claim 1, having the sequence presented as Fmoc-TyrSerPro(modifiedTrp)ThrAsnPhe (SEQ ID NO:2), wherein modifiedTrp is an S-phenylthiocarbamate derivative of tryptophan and Fmoc is linked to the N-terminal α-amino group.

3. A pharmaceutical composition comprising the isolated synthetic peptide of claim 2 and a pharmaceutically acceptable carrier.

4. A method of preventing or treating Staphylococcus infection in a subject, comprising:
administering to the subject a therapeutically effective amount of an isolated synthetic peptide according to claim 1.

5. The method according to claim 4, where said peptide has the sequence presented as Fmoc-TyrSerPro(modifiedTrp)ThrAsnPhe (SEQ ID NO:2), wherein said modifiedTrp is an S-phenylthiocarbamate derivative of tryptophan and Fmoc is linked to the N-terminal α-amino group.

6. A method of preventing or treating a Staphylococcus infection in a subject, comprising:
administering to the subject a therapeutically effective amount of an isolated synthetic amino acid inhibitory of Staphylococcal virulence of the structure, (Fmoc)NH—CHR—COOH, wherein Fmoc is a 9-fluorenylmethoxycarbonyl group and R is a non-polar side chain.

7. The method according to claim 6, wherein said isolated synthetic amino acid is selected from the group consisting of Fmoc-L-Trp(Boc)-OH, Fmoc-D-Trp(Boc)-OH, FMoc-2-aminobenzoic acid, Fmoc-1-amine-cyclohexane carboxylic acid, (R,S)-Fmoc-3-amino-1-cyclohexane carboxylic acid, Fmoc-D-tetrahydroisoquinoline-1-carboxylic acid, Fmoc-4-bromo-L-phenylalanine, Fmoc-4-chloro-L-phenylalanine, Fmoc-5-phenyl-pyrrolidine-2-carboxylic acid, (R)-Fmoc-4-amino-5-phenyl-pentanoic acid, Fmoc-L-His(Trt)-OH, Fmoc-L-1,2,3,4-tetrahydronorharman-3-carboxylic acid, FMoc-2-L-styrylalanine, FMoc-2-L-Lys(Z)-OH, Fmoc-4-methyl-L-phenylalanine and Fmoc-L-Leu-OH.

8. The method according to claim 7, wherein said isolated synthetic amino acid is Fmoc-Trp(Boc)-OH (FTB), wherein modifiedTrp has a BOC group linked to the ring nitrogen.

9. The method according to claim 4 or 6, further comprising administering an antibiotic to said subject.

10. The method according to claim 9, wherein said antibiotic is methicillin or vancomycin

11. The method according to claim 4 or 6, where said Staphylococcus is antibiotic-resistant.

12. The method according to claim 11, where said Staphylococcus is methicillin-resistant.

13. The method according to claim 11, where said Staphylococcus is vancomycin-resistant.

14. A method of preventing a bacterial infection associated with the use of a medical device comprising integrating the isolated synthetic peptide inhibitory of Staphylococcal virulence according to claim 1 with said device and delivering said peptide-containing device to a patient.

15. The method according to claim 14, where said peptide has the sequence presented as Fmoc-TyrSerPro(modifiedTrp)ThrAsnPhe (SEQ ID NO:2), wherein modifiedTrp is an S-phenylthiocarbamate derivative of tryptophan and Fmoc is linked to the N-terminal α-amino group.

16. A method of preventing a bacterial infection associated with the use of a medical device comprising integrating an isolated synthetic amino acid inhibitory of Staphylococcal virulence of the structure,
(Fmoc)NH—CHR—COOH,
wherein Fmoc is a 9-fluorenylmethoxycarbonyl group and R is a non-polar side chain and delivering said amino acid-containing device to the patient.

17. The method according to claim 16, wherein said isolated synthetic amino acid is selected from the group consisting of Fmoc-L-Trp(Boc)-OH, Fmoc-D-Trp(Boc)-OH, FMoc-2-aminobenzoic acid, Fmoc-1-amine-cyclohexane carboxylic acid, (R,S)-Fmoc-3-amino-1-cyclohexane carboxylic acid, Fmoc-D-tetrahydroisoquinoline-1-carboxylic acid, Fmoc-4-bromo-L-phenylalanine, Fmoc-4-chloro-L-phenylalanine, Fmoc-5-phenyl-pyrrolidine-2-carboxylic acid, (R)-Fmoc-4-amino-5-phenyl-pentanoic acid, Fmoc-L-His(Trt)-OH, Fmoc-L-1,2,3,4-tetrahydronorharman-3-carboxylic acid, FMoc-2-L-styrylalanine, FMoc-2-L-Lys(Z)-OH, Fmoc-4-methyl-L-phenylalanine and Fmoc-L-Leu-OH.
